(19) United States
(12) Patent Application Publication
(10) Pub. No.: US 2021/0100754 A1
(43) Pub. Date: Apr. 8, 2021

(54) TRANSDERMAL DRUG DELIVERY SYSTEMS WITH FLUOROSILICONE RELEASE LINERS

(71) Applicant: 3M INNOVATIVE PROPERTIES COMPANY, St. Paul, MN (US)

(72) Inventors: DANIEL CARVAJAL, EDINA, MN (US); RICKY L. LARSON, RIVER FALLS, WI (US); GENE P. McNAMARA, WOODBURY, MN (US); BRUCE D. KLUGE, SOMERSET, WI (US); LARRY D. BOARDMAN, WOODBURY, MN (US)

(21) Appl. No.: 16/074,099

(22) PCT Filed: Jan. 31, 2017
(86) PCT No.: PCT/US2017/015724

§ 371 Date: Jul. 31, 2018

Related U.S. Application Data
(60) Provisional application No. 62/291,782, filed on Feb. 5, 2016.

(51) Int. Cl. A61K 9/70; C08G 77/44; C09J 183/10

(52) U.S. Cl. CPC A61K 9/7069; C09J 183/10; C08G 77/44

(57) ABSTRACT

Fluorosilicone containing release liners for transdermal drug delivery systems, such as transdermal patches, transdermal drug delivery systems containing such release liners, and methods of delivering active pharmaceutical ingredients using the same.

### TECHNICAL FIELD

This disclosure relates to transdermal drug delivery systems, particularly those having fluorosilicone containing release liners.

### BACKGROUND

Transdermal drug delivery systems, such as patches, are an attractive route for delivery of many drugs. Transdermal delivery systems typically contain a substrate with one or more active pharmaceutical ingredients and adhesive disposed thereon. The adhesive is typically covered with a release liner, which serves, for example, to prevent adhesion of unwanted substances to the transdermal delivery system. The release liner can be removed before applying the transdermal drug delivery system to a subject. Finding an appropriate release liner is often challenging.

### SUMMARY

A transdermal drug delivery system can comprise a backing with an active layer disposed on the backing. The active layer can comprise an active pharmaceutical ingredient and an adhesive. A release liner can be disposed over the active layer. The release liner can comprise a substrate, and a release layer disposed on the substrate. The release layer can comprise a blend of (i) a first fluorosilicone polymer that has neither ethylenically unsaturated groups, nor Si—H groups, nor functional groups that are chemically reactive with ethylenically unsaturated groups or Si—H groups, and (ii) an at least partially cured reaction product of a second fluorosilicone polymer that has at least two cross-linkable functional groups per polymer chain and a non-fluorinated silicone polymer that comprises at least two cross-linkable functional groups per polymer chain and does not contain any fluorine atoms. At least a portion of the release layer can be in contact with the active layer. The active pharmaceutical ingredient can contain one or more functional groups that are chemically reactive with the cross-linkable functional groups of the second fluorosilicone polymer, the cross-linkable functional groups of the non-fluorinated silicone polymer, or both.

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context.

Some terms used in this application have special meanings, as defined herein. All other terms will be known to the skilled artisan, and are to be afforded the meaning that a person of skill in the art at the time of the invention would have given them.

“Independently selected” when used in reference to the identify of one or more variable elements means that each occurrence of any of the variable elements may have the same or different identity, within the specified limitations, regardless of the identity of any other occurrence of the reference element. Thus, if there are two occurrences of element “E,” and element E can be independently selected from identity A or identity B, each of the two occurrences of E can be either A or B, in any combination (e.g., AA, AB, BA, or BB).

“Fluorocarbon” refers to an aliphatic hydrocarbon group or radical, as called for by the context, wherein at least one hydrogen atom is replaced by a fluorine atom.

“Perfluorocarbon” refers to a fluorocarbon wherein virtually every hydrogen atom is replaced by a fluorine atom, without excluding the possibility that some of the hydrogen atoms have been replaced with chlorine, bromine, or iodine. For example, when a polymer containing a perfluorocarbon group is manufactured by way of a polymerization reaction with chlorotrifluoroethylene, some of the hydrogen atoms may be replaced with chlorine instead of fluorine.

“Fluoroether” refers to an ether moiety wherein at least one hydrogen atom in the ether moiety is replaced by a fluorine atom.

“Perfluoroether” refers to a fluoroether wherein virtually every hydrogen atom is replaced by a fluorine atom, without excluding the possibility that some of the hydrogen atoms have been replaced with chlorine, bromine, or iodine. For example, when a polymer containing a perfluoroether group is manufactured by way of a polymerization reaction with chlorotrifluoroethylene, some of the hydrogen atoms may be replaced with chlorine instead of fluorine.

A transdermal drug delivery system can include a backing layer, an active layer disposed on the backing layer, and a release liner disposed over the active layer. The active layer can include an active pharmaceutical ingredient and an adhesive. The release liner can include a substrate and a release layer disposed on the substrate such that at least a portion of the release layer is in contact with at least a portion of the active layer, and particularly with at least a portion of the adhesive.

The substrate of the release liner can be any suitable substrate. Suitable substrates include polyesters, such as polyethylene terephthalate, paper, such as kraft, super calandered kraft, glassine, polyethylene or polypropylene coated kraft papers, polyethylene, such as low density polyethylene, high density polyethylene, low density linear polyethylene, nylon, cellulose acetate, polyurethane, ethyl cellulose, polyvinyl chloride, polyvinylidine chloride, ethylene vinyl acetate copolymer, or composites of any of these materials. The substrate is typically in the form of a thin film.

The release layer, which is disposed on the substrate of the release liner, can include a blend of a first fluorosilicone polymer that has neither ethylenically unsaturated groups, nor Si—H groups, nor functional groups that are chemically reactive with ethylenically unsaturated groups or Si—H groups, and an at least partially cured reaction product of a second fluorosilicone polymer that has at least two cross-linkable functional groups per polymer chain and a non-fluorinated silicone polymer that comprises at least two cross-linkable functional groups.

The first fluorosilicone polymer does not contain ethylenically unsaturated groups and also does not contain Si—H groups or groups that chemically react with ethylenically unsaturated groups or Si—H groups. However, other functional groups that do not readily react with ethylenically unsaturated groups or Si—H groups, such as epoxy groups, ether groups, fluoroether groups, or perfluoroether groups, can be included. Thus, the first fluorosilicone polymer can have a siloxane backbone and at least one pendant fluorinated group. The pendant fluorinated group can be a fluoroalkyl or fluoroether group, which can be chemically bound to the siloxane backbone. The fluoroalkyl or fluoroether group can be chemically bound to the siloxane group either directly or by way of a linking group.

When the pendant fluorinated group is perfluoroalkyl, it can have from one to six carbon atoms, such as from two to six carbon atoms, or from two to four carbon atoms. One exemplary perfluoroalkyl group is C₄F₉. When one or more linking groups are present, the linking groups are commonly independently selected from alkylene groups having at least two carbon atoms. Ethylene is common. Thus, the pendant fluorinated group can include a C₄F₉ group covalently bound to the siloxane backbone by way of an ethylene linking group.

The pendant fluorinated group can also be a fluoroether group. Suitable fluoroether groups are described in WO 2014/193654

The first fluorosilicone polymer can be prepared from a fluorosilicone polymer having ethylenically unsaturated groups or Si—H groups or groups that react with ethylenically unsaturated groups or Si—H groups by converting all of those functional groups into other functional groups that are not ethylenically unsaturated groups or Si—H groups and do not react with ethylenically unsaturated groups or Si—H groups. For example, ethylenicaly usaturated groups can react with monohydride functional group containing silicones, such as pentamethyldisiloxane, to form the first fluorosilicone polymer. Particular first fluorosilicone polymers can be prepared by reaction of vinyl-functional fluorinated polyorganosiloxane polymers. Suitable vinyl-functional fluorinated polyorganosiloxane polymers for preparation of the first fluorosilicone polymer include fluorinated vinyl functional polydimethylsiloxane. Suitable vinyl-functional fluorinated polyorganosiloxane polymers for preparation of the first fluorosilicone polymer are commercially available, for example, under the trade name SYL-OFF Q2-7785 and SYL-OFF 7786 (Dow Corning Corp., Midland, Mich., USA).

The reaction of the vinyl functionalized fluorinated polyorganosiloxane can take place in an organic solvent. Any suitable solvent can be used. Suitable solvents include liquid alkanes, such as heptane, hexane, and cyclohexane, dichloromethane, toluene, benzene, xylenes, tetrahydrofuran, and the like. Pentamethyldisiloxane, or a similar agent that can react with the ethylenically unsaturated groups in the vinyl functionalized fluorinated polyorganosiloxane to form unreactive groups can be added. A catalyst, such as a catalyst suitable for hydrosilyation, can also be used. Such catalysts are known, and examples include the platinum hydrosilylation catalyst that is formulated with SYL-OFF Q2-7785 and SYL-OFF 7786. The reaction can take place at room temperature, but is more commonly done at elevated temperature, such as temperatures higher than 50° C., and most commonly at or about the boiling temperature of the solvent used.

Other examples of first fluorosilicone polymers include polymers available under the trade designation FMS-141 (Dow Corning) and those described as non-functional fluorinated silicone polymers in WO2015/095173.

The second fluorosilicone polymer has cross-linkable groups. The cross-linkable groups are usually ethylenically unsaturated groups, but other groups can also be used.

The second fluorosilicone polymer has at least two cross-linkable groups per polymer chain. By this, it is meant that there is an average of at least two cross-linkable groups, typically ethylenically unsaturated, groups per polymer chain. While in most cases all of the polymer chains will have at least two cross-linkable groups, typically ethylenically unsaturated groups, it is possible that some polymer chains will have fewer than two and some will have greater, so long as the average number of cross-linkable groups, which are typically ethylenically unsaturated groups, is at least two per polymer chain

Any fluorosilicone polymer having the requisite amount of cross-linkable groups, such as ethylenically unsaturated groups, can serve as the second fluorosilicone poylymer. When the cross-linkable groups are ethylenically unsaturated groups, the at least two ethylenically unsaturated groups are often present as two terminal ethylenically unsaturated groups, but this is not necessary unless specifically called for. It is also possible for one or both of the at least two ethylenically unsaturated groups to be pendant from the polymer chain rather than at the ends.

Exemplary second fluorosilicone polymers are triorganosiloxy end-capped polydiorganosiloxanes. Such polymers have R₃SiO groups at the termini and R₂SiO groups in the remainder of the polymer chain, wherein each R is independently hydrocarbon, ethylenically unsaturated hydrocarbon, fluorocarbon or fluoroether, and wherein at least two R groups are ethylenically unsaturated hydrocarbon and at least one R group is fluorocarbon or fluoroether group. The hydrocarbon and fluorocarbon usually have from 1 to 6 carbon atoms each. When one or more R groups are fluorocarbon radicals, the fluorocarbon radicals are often perfluorocarbon radicals. When one or more R groups are fluoroether, the fluoroether is often perfluoroether.

Thus, the second fluorosilicone polymer can be a fluoroorganopolysiloxane polymer having a siloxane backbone and pendant perfluoroalkyl or perfluoroether groups covalently bonded to a linking group, the linking group being covalently bonded to the siloxane backbone. The perfluoroalkyl groups often have from 1 to 6 carbon atoms, such as 1 carbon atom, 2 carbon atoms, 3 carbon atoms, 4 carbon atoms, 5 carbon atoms, or 6 carbon atoms. The linking group is often an ethylene group. Particular second polymers have siloxane backbones with ethylene linking groups covalently bonded to both the siloxane backbone and to a pendant perfluorobutyl group. Ether-containing linking groups can also be used. Perfluoroether groups can be used as the pendant groups, either in addition to or in combination with perfluoroalkyl groups.

The ethylenically unsaturated groups can be independently selected from groups having the formula —(CH₂)ₘCH═CH₂, wherein m is, in some cases, 0, 1, 2, 3, or 4. When m is 0, the ethylenically unsaturated group is vinyl. In many cases, m is 4 and the ethylenically unsaturated group is 5-hexenyl. In other cases, m can be from 0 to 15.

When the second fluorosilicone polymer contains ethylenically unsaturated groups as the cross-linkable groups, then the vinyl equivalent weight of the second fluorosilicone polymer can be no greater than 50,000 grams per equivalent, such as no greater than 30,000 grams per equivalent, or no greater than 25,000 grams per equivalent. In some cases the fluorosilicone polymer can have a vinyl equivalent weight of at least 1,000 grams per equivalent, such as at least 2,000 grams per equivalent or at least 3,000 grams per equivalent. In many cases the vinyl equivalent weight is from 1,500 to 10,000 grams per equivalent, such as from 2,000 to 8,000 grams per equivalent or 3,000 to 7,000 grams per equivalent.

When the second fluorosilicone polymer contains other cross-linkable groups, such as Si—H groups, the amount of cross-linkable groups can be defined by a cross-linkable group equivalent weight, which is analogous to a vinyl equivalent weight except that it compares the grams of polymer to equivalents of cross-linkable groups (instead of grams of polymer to equivalents of vinyl groups). Where applicable, the cross-linkable group equivalent weight can be at least 1,000 grams per equivalent, such as at least 2,000 grams per equivalent or at least 3,000 grams per equivalent. In many cases the cross-linkable group equivalent weight is from 1,500 to 10,000 grams per equivalent, such as from 2,000 to 8,000 grams per equivalent or 3,000 to 7,000 grams per equivalent.

A number of useful, commercially available, functional fluorosilicone polymers may be used as the second fluorosilicone polymer and are available from Dow Corning Corp. (Midland, Mich.) under the SYL-OFF series of trade designations including, e.g., SYL-OFF Q2-7785 and SYL-OFF 7786 and can be used as the second fluorosilicone polymer. Other functional fluorosilicone polymers that can be used as the second fluorosilicone polymer are commercially available from General Electric Co. (Albany, N.Y.), and Wacker Chemie (Germany). Additional functional fluorosilicone polymers are described as component (e) at column 5, line 67 through column 7, line 27 of U.S. Pat. No. 5,082,706 (Tangney).

The non-fluorinated silicone polymer contains at least two cross-linkable groups, such as at least two ethylenically unsaturated groups, per polymer chain. When the cross-linkable groups are ethylenically unsaturated groups, then the non-fluorinated silicone polymer typically has a vinyl equivalent weight that is no greater than 20,000 grams per equivalent, such as no greater than 15,000 grams per equivalent, or no greater than 10,000 grams per equivalent. In many cases, the non-fluorinated silicone polymer has a vinyl equivalent weight of at least 250 grams per equivalent, such as at least 500 rams per equivalent or at least 1,000 grams per equivalent. Exemplary non-fluorinated silicone polymers can have a vinyl equivalent weight of 500 to 5,000 grams per equivalent, such as 750 to 4,000 grams per equivalent, or 1,000 to 3,000 grams per equivalent.

When the non-fluorinated silicone polymer contains other cross-linkable groups, such as Si—H groups, the amount of cross-linkable groups can be defined by a cross-linkable group equivalent weight, which is analogous to a vinyl equivalent weight except that it compares the grams of polymer to equivalents of cross-linkable groups (instead of grams of polymer to equivalents of vinyl groups). Where applicable, the cross-linkable group equivalent weight can be at least 1,000 grams per equivalent, such as at least 2,000 grams per equivalent or at least 3,000 grams per equivalent. In many cases the cross-linkable group equivalent weight is from 1,500 to 10,000 grams per equivalent, such as from 2,000 to 8,000 grams per equivalent or 3,000 to 7,000 grams per equivalent.

Suitable non-fluorinated silicone polymers are commercially available, for example, under the trade designations DMS-V03, DMS-V05, DMS-V21, DMS-V22, DMS-V25, and DMS-V41 from Gelest, Inc. Other commercially available polymers that are suitable include DOW 2-7120 and DOW 7850, available from Dow Corning Corp., VMS-T11 and SIT7900, available from Gelest, Inc., SILMER-VIN 70, available from Siltech Corp., and 2,4,6,8-tetramethyl-2-4-6-8-tetravinylcyclotetrasiloxane, available from Aldrich (St. Louis, Mo., USA).

The ratio of the cross-linkable group equivalent weight or vinyl equivalent weight of the second fluorosilicone polymer over the vinyl equivalent weight of the non-fluorinated silicone polymer can be varied in order to control the release force, that is, the force needed to remove the release layer from the rest of the transdermal drug delivery system, and particularly from the adhesive component of the transdermal drug delivery system. In particular, lower release forces can be obtained as this ratio is increased. Appropriate ratios of the vinyl equivalent weight of the second fluoropolysilicone to the non-fluorinated silicone polymer is at least 1, such as at least 2, or at least 3. In some embodiments, the ratio is from 2 to 6 such as from 3 to 5.

A cross-linking agent can be used along with the other components of the release layer in order to aid in at least partially curing the release layer. Any suitable cross-lining agent can be used. Typically, organohydrogensiloxane cross-linking agents are used although this is not required unless otherwise specified. Suitable cross-linking agents include those available under trade designations SYL-OFF 7488, SYL-OFF 7678, SYL-OFF Q2-7560, and SYL-OFF 7561 from Dow Corning Corp. The cross-linking agents described in U.S. Pat. Nos. 5,082,706 (Tangney) and 5,578,381 (Hamada et al.) can also be used. Most often, the cross-linking agent will include hydride functional groups. Notably, cross-linking agents are not needed in all cases because some polymers used in the release layer can at least partially cure without a cross-linking agent, for example, upon exposure to ultraviolet light or elevated temperature.

The cross-linking agent can be used in any suitable amount to form the at least partially cured reaction product. When the cross-linking agent contains hydride functional groups, the cross-linking agent can be used in an amount such that the ratio of hydride groups in the cross-linking agent to ethylenically unsaturated groups in the polymers is no less than 1, such as no less than 1.1, no less than 1.15, or no less than 1.2. The ratio of hydride groups in the cross-linking agent to ethylenically unsaturated groups in the polymers can also be no greater than 4, such as no greater than 3.5, or no greater than 3.

The release layer can be coated on the substrate of the release liner by any known means. Typically, solvent casting is used. Solvent casting can be, for example, by rod coating, gravure coating, drop coating, or spin coating.

In an exemplary solvent casting process, the components of the release layer, along with at least cross-linking agent if used, can be added to a suitable organic solvent. A suitable platinum hydrosilation catalyst, such as divinyltetramethyldisiloxane platinum, available from Gelest, and inhibitor, such as diallyl maleate, can also be added to the organic solvent. The organic solvent can be any solvent that dissolves or disperses the components. Typically, ethyl acetate, acetone, liquid alkanes such as heptanes, hexanes, or cyclohexane, tetrahydrofuran, benzene, toluene, xylenes, or some combination thereof is used. A mixture of ethyl acetate and hexanes or ethyl acetate and heptanes is most common.

Once the components are dissolved or dispersed in the organic solvent, the components can then be coated onto the substrate of the release liner, for example, by rod coating, gravure coating, drop coating, or spin coating.

The coating on the substrate can then be at least partially cured to form the release layer. Curing can be initiated by any suitable method, such as by irradiation, for example with UV or visible light, by heating, or in some cases by allowing the coating to stand for a sufficient amount of time.

The release layer can be fully cured, for example, such that further curing does not alter the relevant properties of the release layer, but this is not necessary in all cases. Instead, the coating can be cured until the desired properties, such as release properties, are obtained. In some cases, the release layer is cured such that at least 75% of all of the cross-linkable groups, such as ethylenically unsaturated groups, are reacted. For example, the release layer can be cured until at least 80%, at least 85%, at least 90%, at least 95%, at least 98%, or at least 99% of the cross-linkable groups, such as ethylenically unsaturated groups, are cross-linked.

The release layer typically comprises at least 50 wt. % of the non-fluorinated silicone polymer based on the combined weight of the non-fluorinated silicone polymer and the second fluorosilicone polymer. Further, the release often layer comprises 65 to 97 wt. % of the non-fluorinated silicone polymer based on the combined weight of the non-fluorinated silicone polymer and the second fluorosilicone polymer.

The release layer can be a release layer described in WO 2015/095173, and can be made and coated onto a substrate by the methods described therein.

The backing of the transdermal delivery system can be formed of any suitable material, such as a flexible film. Examples of flexible films that can be used include those flexible films made of polymers such as polypropylene, polyethylene, for example low density polyethylene, linear low density polyethylene, metallocene polyethylene, or high density polyethylene, polyvinyl chloride, polyester, such as polyethylene terephthalate, polyvinylidine chloride, ethylene vinyl acetate copolymer, polyurethane, cellulose acetate, or ethyl cellulose. Coextruded multilayer polymeric films, such as those described in U.S. Pat. No. 5,783,269 can also be suitable. The backing can include more than one layer, for example, a polyethylene terephthalate-aluminum-polyetheylene layered structure, or a polyethylene terephthalate-ethylene vinyl acetate copolymer layered structure can be used. Foam and tape backings can also be suitable. Examples include closed cell polyolefin films, available under the trade designations 3M™ 1777 Foam Tape and 3M™ 1799 Foam Tape (both available from 3M Company, St. Paul, Minn., USA). Polymer blends, such as those of one or more types of polyethylene, as well as composites of polyethylene, can be used. Polyurethanes are also suitable in many cases. The backing can, in some cases, be translucent or transparent. The backing can also include one or more additive, depending on the desired properties of the particular backing. Additives can include one or more of tackifiers, plasticizers, colorants, radical scavengers, and anti-oxidents.

Some backings are impermeable or have an impermeable layer, which is typically disposed facing the adhesive, in order to substantially or completely prevent migration of the active pharmaceutical ingredient towards or into the backing. Impermeable layers or backings often include films having one or more layers of polyethylene terephthalate or aluminum. Impermeable layers or backings can also be films having plasma-deposited amorphous glass layers, such as those described in WO 2011/066493, and films having inorganic barrier layers, such as those described in U.S. Pat. Pub. 2004/202708.

The backing can be any suitable thickness; typically, the thickness of the backing is sufficient to make the transdermal delivery system manipulatable by a human. Often the thickness is 10 microns or greater; such as 20 microns or greater, 40 microns or greater, 1 mm or greater, or 2 mm or greater. In many cases, the thickness is less than 2 mm, such as less than 1 mm, or less than 150 microns.

The adhesive is typically suitable for contacting skin, particularly mammalian or human skin. Thus, in some cases the adhesive does not contain materials such as skin irritants, toxins, or the like, or when such materials present, they are present in a sufficiently low concentration that they would not preclude contacting the adhesive with the skin of a typical mammal, particularly a human, for sufficient time to deliver all or some of the active pharmaceutical ingredient.

The adhesive is usually a pressure sensitive adhesive, most commonly a pressure sensitive adhesive that is capable of securely but releasable adhering to skin, particularly mammalian or human skin. In some cases, more than one type of adhesive, such as pressure sensitive adhesive, can be present, for example, multiple layers, each of which may be disposed over all or a part of the backing and each of which may include the same or different adhesives, can be present. In such cases, each of the adhesives can be independently selected from suitable adhesives. Suitable adhesives include acrylates, which may have acrylate, methacrylate, or most commonly both acrylate and methacrylate components, silicones, polyisobutylenes, rubber, which can be synthetic or natural, and copolymers or mixtures thereof.

The transdermal delivery system described here is particularly well suited for silicone or polysiloxane adhesives. This is so because many silicone adhesives adhere strongly to most release liners such that most release liners cannot be readily removed. The release liners having release layers as described herein, however, can be removed from silicone adhesives without the need for unacceptably high force. While the release layers described herein can provide good results with adhesives other than silicone adhesives, such other adhesives might also give acceptable results when used with release layers that are less expensive than those disclosed herein.

The silicone or polysiloxane adhesives are often pressure sensitive adhesives. Silicone pressure sensitive adhesives typically include a silicone polymer or gum and a tackifying resin. The silicone adhesive can be prepared by crosslinking the polymer with the resin in a condensation reaction to produce a three-dimensional silicate structure. The condensation reaction typically takes place in an organic solvent. The physical properties of the silicone adhesive can be modified by varying the ratio of polymer to resin. Capped or amine-compatible silicones can be used to decrease drug degradation. Suitable silicone adhesives are known in the art, and are described, for example, in U.S. Pat. Nos. 4,591,622, 4,584,335, 4,585,836, and 4,655,767. Suitable silicone adhesives are available commercially, and include those sold under the trade designation BIO-PSA by Dow Corning Corporation (Midland, Mich., USA).

The transdermal delivery systems described herein can use an acrylate adhesive. When an acrylate is used, it is typically a copolymer. Acrylate adhesives typically have an inherent viscosity of 0.2 dL/g and comprise one or more polymerized primary monomers and, optionally, one or more polymerized polar comonomers. Primary monomers that are suitable for use include alkyl acrylates having 4 to 12 carbon atoms in the alkyl group and alkyl methacrylates having 4 to 12 carbon atoms in the alkyl group. Examples of specific alkyl groups, any of which can be used with either an acrylate or a methacrylate, include n-butyl, n-pentyl, n-hexyl, isoheptyl, n-nonal, n-decyl, isohexyl, 2-ethyloctyl, isooctyl (also known as 2-methyl heptyl), 2-ethylhexyl, and cyclohexyl. One or more of these can be used alone or in combination with each other. Polar monomers that are suitable include those having hydroxyl, amide, carboxylic acid, sulfonic acid, or phosphonic acid functional groups. Examples include acrylamide, methacrylamide, N-vinyl-2-pyrrolidone, 2-hydroxyethylacrylate, 2-hydroxyethylmethacrylate, hydroxyproplyacrylate, 2-hydroxyethylmethacrylate, hydroxypropylacrylate, acrylic acid, methacrylic acid, pyrrolidonyl ethyl acrylate, and alkoxyethyl acrylates such as 2-carboxyethyl acrylate. Acrylamide is most common. In most cases, the weight of all of the polymerized polar monomer, such as acrylamide, will not exceed 40% of the weight of all of the monomers in the polymer; additional polar monomer can, in some cases, cause the adhesive to be too firm. Most commonly, the polar monomers, if used, are present from 1% to 20% of the weight of the entire polymer.

Acrylate copolymers can also include polymerized optional monomers. When present, such optional monomers are included in amounts that will not eliminate the tackiness of the adhesive. Such optional monomers are instead used in amounts that improve the performance of the adhesive, reduce the cost, or serve some other purpose. Examples of optional monomers include vinyl esters such as vinyl acetate, vinyl chloride, vinylidine chloride, styrene, and macromonoers that are useable with the primary and polar monomers. Such macromonomers include polymethylmethacrylate, styrene/acrylonitrile copolymer, polyether, and polystyrene. Examples of macromonomers and their preparation are known and are described, for example, in U.S. Pat. No. 4,963,776.

Polyisobutylene adhesives can also be used. Suitable polyisobutylene adhesives are known in the art and are described, for example, in U.S. Pat. No. 5,380,760.

Blends of any of the foregoing adhesives may also be used. In particular, silicone or polysiloxane adhesives can be used as a blend with other adhesives or polymers. For example, when polyacrylate or polyisobutylene adhesives are used, they are most commonly used in a blend with silicone or polysiloxane adhesives rather than by themselves. Examples of blends that can be employed are blends of polysiloxane with polyacrylate, polysiloxane with polyisobutylene, and polysiloxane with polyisoprene-polystyrene. Particular adhesive blends that can be suitable are known in the art and are described, for example, in U.S. Pat. Nos. 5,656,286, 5,958,446, 6,024,976, and 6,638,528.

The adhesive can have any suitable thickness on the backing. The thickness can be at least 10 microns, at least 20 microns, at least 30 microns, or at least 40 microns. The thickness can also be less than 2 mm, less than 1 mm, or less than 150 microns.

While it is possible to use any active pharmaceutical ingredient suitable for transdermal delivery, the transdermal delivery system described herein is particularly designed for those active pharmaceutical ingredients that contain amines, particularly secondary and tertiary amines.

Examples of some specific active pharmaceutical agents that can be used with the transdermal delivery system described herein include buprenorphine, clonidine, fentanyl, granisetron, methyl phenidate, oxybutynin, rivastigmine, rotigotine, scopolamine, selegiline, nicotine, sumatriptan, capsaicin, diclofenac epolamine, lidocaine, etidocaine, ropivacaine, indapamide, apomorphine, propylnorapromorphine, salbuterol, lisuride, dihydroergotamine, pergolide, terguride, proterguride, propranolol, imipramine, guanethidine, cyproheptadine, olanzapine, and diclofenac. Those active pharmaceutical agents are particularly useful because they contain at least one amine, typically a secondary or tertiary amine, and have been reported as useful for transdermal delivery.

Active pharmaceutical ingredients with amine functional groups, particularly secondary and tertiary amine functional groups, can cause problems with prior art fluorosilicone or fluoropolymer release liners. These problems can be particularly difficult to overcome when such release liners are used in conjunction with silicone adhesives. Specifically, the force needed to remove prior art fluorosilicone release liners from transdermal delivery systems containing amines can either be unacceptably high, or can increase over time until it is unacceptably high. When this force is unacceptably high, it can be unacceptably difficult to remove the release liner from the transdermal delivery system. One possible mechanism that can cause this problem is a chemical interaction between the pharmaceutically active agent bearing amine functional groups and the cross-linking groups in the prior-art fluorosilicone or fluoropolymer release layers. This mechanism is suspected because amines are well known to undergo a variety of chemical reactions with cross-linking groups, particularly ethylenically unsaturated groups. Further, the problem can be more pronounced when secondary or tertiary amines, which are typically more nucleophilic than primary amines, are present in the pharmaceutically active agent. However, additional or other mechanisms may be causing this phenomenon as well.

This disclosure provides, among other things, a way to lessen, ameliorate, mitigate, or in some cases solve, the above-mentioned problem. Surprisingly, when the release layer disclosed herein is used, the force needed to remove the release liner is not observed to increase to the point where it is unacceptably difficult to remove the release liner from the transdermal delivery system.

Thus, the force needed to remove the release liner from the transdermal delivery system described herein is typically not be unacceptably high. Further, the force is typically not too low, because a force that is too low can be representative of insufficient adhesion between the release liner and the rest of the transdermal delivery system. A force that is too low can result in unintentional separation of the release liner before use. Thus, the force needed to remove the release liner from the transdermal delivery system is typically from 5 to 50 g/25 mm, such as from 10 to 40 g/25 mm, when tested according to the method described in the Examples section of this disclosure. Even after storage, such as at 25° C. and 60% relative humidity, or even at 40° C. and 75% relative humidity, for 4 weeks, 6 weeks, 8 weeks, 12 weeks, 6 months, 1 year, or even 2 years, the force needed to remove the release liner from the transdermal delivery system is typically from 5 to 50 g/25 mm, such as from 10 to 40 g/25 mm, when tested according to the method described in the Examples section of this disclosure.

The active layer can have any suitable disposition of the adhesive and the pharmaceutical active agent. For example, the active layer can be arranged such as to form a drug reservoir. The drug reservoir can be an area between the backing and all or part of the adhesive in which some or all of the active pharmaceutical agent is present. The surface area of a drug reservoir, if present, can be the same as the surface area of the transdermal delivery system, but is typically smaller than the surface area of the transdermal delivery system such that some portion of the transdermal delivery system can be handled by a user without contacting the adhesive above the drug reservoir, and such that the drug reservoir can be surrounded by a rim of adhesive on all sides. This configuration can be helpful in securing the transdermal delivery system to the surface of skin, such as mammalian skin and particularly human skin. In most cases, the surface area of the drug reservoir can be at least 1.0 cm², such as at least 5 cm². Most commonly, the drug reservoir will have a surface area that is less than 100 cm², such as less than 40 cm². The drug reservoir is often no more than 2 mm thick, such as no more than 1 mm thick or no more than 150 microns thick.

The active layer, including the reservoir, and the backing can together constitute a transdermal patch. Various arrangements of reservoirs on such patches are possible. Examples include those containing gelled or liquid reservoirs, for example as described in U.S. Pat. No. 4,834,979; patches containing matrix reservoirs attached to the skin by adjacent adhesive, for example as described in U.S. Pat. No. 6,004,578; and patches containing drug in a matrix of adhesive, for example as described in U.S. Pat. Nos. 6,365,178, 6,024,976, 4,751,087, and 6,149,935.

In another configuration, which is an alternative to the drug reservoir, the active pharmaceutical ingredient can be present in a matrix that is a distinct layer and is adhered to at least a portion of the adhesive. The matrix itself can be adhesive or non-adhesive. When the matrix is non-adhesive or is insufficiently adhesive to securely affix the transdermal delivery system to skin, attachment can be effected by a rim of adhesive, such as any of the adhesives described above, around the matrix.

It is also possible for the drug to be present in the form of solid particles that are embedded in the adhesive or are on the surface of the adhesive. In many cases, the particles are hydrophilic such that contact with aqueous fluid at the surface of treated skin will cause them to dissolve or disintegrate thereby releasing the active pharmaceutical ingredient into the skin.

The active pharmaceutical ingredient can be present in the adhesive, which can be any of the adhesives discussed herein but is most commonly a silicone adhesive. Depending on the nature of the particular active pharmaceutical ingredient, it can either be dissolved in the adhesive, or it can be present in the form of dispersed particles. Suitable methods for applying active pharmaceutical ingredients to adhesives have been disclosed in U.S. Pat. Pub. No. 2003/054025 and U.S. Pat. No. 5,688,523.

The active layer most often includes only one layer of adhesive, but in some configurations it is possible for two or more discrete layers of adhesive to be present. For example, a first adhesive layer that contacts the release liner can function as a skin-contacting adhesive that secures the transdermal delivery system to the skin as well as a second adhesive layer that is between the first adhesive layer and the backing. The second adhesive layer can contain the pharmaceutically active ingredient and serve as the drug reservoir.

In this configuration, it is possible for the one or both of the two adhesive layers to serve additional purposes beyond affixing the transdermal delivery system to the skin. For example, the first adhesive layer can be rate-controlling in that it serves to control the rate of delivery of the pharmaceutically active ingredient to the skin. This control in the rate of the drug can be achieved by selecting a first adhesive and second adhesive that have different affinities for the drug, that provide different permeation rates of the drug, that have different thicknesses, or a combination of the foregoing. In this case, the first adhesive layer typically has a lower affinity for the pharmaceutically active ingredient than the second adhesive layer. Thus, the diffusion of pharmaceutically active ingredient out of the second adhesive, or reservoir, is retarded compared to a configuration wherein the first adhesive layer is identical to the second adhesive layer. The rate of diffusion out of the transdermal delivery system can be further controlled by changing the thickness of the first adhesive layer. Generally, a thicker first adhesive layer will correspond to slower diffusion and thus a longer delivery time. Typically, the thickness of the first adhesive layer greater than 25 microns, greater than 50 microns, or greater than 100 microns. The thickness of the first adhesive layer can also be less than 150 microns or less than 120 microns. The thickness of the second adhesive layer can be greater than 10 microns, such as greater than 20 microns. The thickness of the second adhesive layer can be less than 50 microns such as less than 40 microns.

At least one solubilizing agent can also be present in the active layer. When present, the at least one solubilizing agent is typically added to the adhesive to increase the solubility of the drug in the adhesive. Any suitable solubilizing agent can be used. Examples include butylene glycol, diethylene glycol methyl ester, dipropylene glycol, propylene glycol, and mixtures of the foregoing. When present, the solubilizing agent is often used in the active layer in any suitable amount. Suitable amounts can be 1 wt. % or greater, such as 2 wt. % or greater, or 3 wt. % or greater, based on the weight all of the components of the active layer. The solubilizing agent can also be present in no more than 10 wt. %, no more than 9 wt. %, no more than 8 wt. %, or no more than 6 wt. % of the active layer, based on the weight of all of the components of the active layer. Solubilizing agents are not used in all cases. Active pharmaceutical agents are not necessarily dissolved in the adhesive, and even when they are, some active pharmaceutical agents are sufficiently soluble in an adhesive such that solubilizing agents are not needed.

At least one permeation enhancer can also be added to the active layer. When present, any suitable permeation enhancer can be used to increase the permeation of the pharmaceutically active agent into the skin. Suitable permeation enhancers include levulinic acid, dipropylene glycol, oleic acid, and combinations thereof When present, the permeation enhancer can be present in the active layer in any suitable amount. For example, the permeation can be present in amounts of 4 wt. % or greater, 5 wt. % or greater, 7 wt. % or greater, or 10 wt. % or greater based on the total weight of the active layer. The permeation can also be present in amounts less than 25 wt. %, less than 22 wt. %, less than 20 wt. %, or less than 15 wt. % based on the total weight of the active layer.

The transdermal delivery system can be prepared by any suitable method of preparing transdermal delivery systems or patches. For example the adhesive, active pharmaceutical agent, solubilizer if used, and permeation enhancer if used can be combined with an organic solvent. Typical organic solvents used for this purpose include ethyl acetate, isopropanol, methanol, acetone, 2-butanone, ethanol, toluene, alkanes, such as hexanes, or mixtures thereof. This combination can then be used to cast a film of the active layer on the release liner. A backing can then be placed over the active layer. Alternatively, an active layer film can be cast on the backing and a release layer placed over the active layer.

The transdermal delivery system can be in the form of an article, such as a tape, patch, sheet, dressing, or other form that is suitable in the art. Most commonly, the transdermal delivery system is in the form of a patch.

In use, the release liner is removed from the transdermal delivery system thereby exposing the active layer. The active layer is contacted to the skin and securely adhered thereto, typically by way of the adhesive. If needed, medical tape or a similar securement system can be used to affix the transdermal delivery system to the skin. The transdermal delivery system remains in contact with the skin for a sufficient time to deliver the active pharmaceutical ingredient. The skin contact time can vary, but is usually from lminute to 14 days. The skin contact time is, in many cases, 7 days (weekly dosing), 3 to 4 days (twice-weekly dosing), or 1 day (daily dosing). In other cases, the skin contact time is from 1 minute to 1 hour. In some cases, the skin contact time is at least 1 minute, at least 5 minutes, at least 15 minutes, or at least 30 minutes. The skin contact time can also be no more than 1 hour, no more than 45 minutes, no more than 30 minutes, or no more than 20 minutes. The skin contact time is sufficient to deliver at least some of the active pharmaceutical ingredient, but it need not be sufficient to deliver all of the active pharmaceutical ingredient. It is possible that some active pharmaceutical ingredient will remain in the transdermal delivery system even after removal from the skin.

### List of Exemplary Embodiments

The following list of embodiments is provided to further illustrate particular features of the disclosure, and is not intended to be limiting.

1. A transdermal drug delivery system comprising
a backing;
an active layer disposed on the backing, the active layer comprising:
   an active pharmaceutical ingredient containing at least one amine functional group, and
   an adhesive;
a release liner disposed over the active layer, the release liner comprising:
   a substrate, and
   a release layer disposed on the substrate; wherein
at least a portion of the release layer is in contact with the active layer;
the release layer comprising a blend of
   a first fluorosilicone polymer that has neither ethylenically unsaturated groups, nor Si—H groups, nor functional groups that are chemically reactive with ethylenically unsaturated groups or Si—H groups, and
an at least partially cured reaction product of
   a second fluorosilicone polymer that has at least two cross-linkable functional groups per polymer chain, and
   a non-fluorinated silicone polymer that comprises at least two cross-linkable functional groups per polymer chain and does not contain any fluorine atoms.
      2. The transdermal drug delivery system of embodiment 1, wherein the cross-linkable functional groups of the second fluorosilicone polymer comprise ethylenically unsaturated groups.
      3. The transdermal drug delivery system of any of the preceding embodiments, wherein the release layer comprises at least 50 wt. % of the non-fluorinated silicone polymer based on the combined weight of the non-fluorinated silicone polymer and the second fluorosilicone polymer.
      4. The transdermal drug delivery system of any of the preceding embodiments, wherein the release layer comprises 65 to 97 wt. % of the non-fluorinated silicone polymer based on the combined weight of the non-fluorinated silicone polymer and the second fluorosilicone polymer.
      5. The composition of any of the preceding embodiments, wherein the blend comprises no greater than 30 parts by weight of the first fluorosilicone polymer per 100 parts by weight of the combined weight of the non-fluorinated silicone polymer the second fluorosilicone polymers.
      6. The transdermal drug delivery system of any of the preceding embodiments, wherein the cross-linkable functional groups of the non-fluorinated silicone polymer comprise ethylenically unsaturated groups.
      7. The transdermal drug delivery system of any of the preceding embodiments, wherein the active pharmaceutical ingredient comprises at least one of buprenorphine, clonidine, fentanyl, granisetron, methyl phenidate, oxybutynin, rivastigmine, rotigotine, scopolamine, selegiline, nicotine, sumatriptan, capsaicin, diclofenac epolamine, lidocaine, etidocaine, ropivacaine, indapamide, apomorphine, propylnorapromorphine, salbuterol, lisuride, dihydroergotamine, pergolide, terguride, proterguride, propranolol, imipramine, guanethidine, cyproheptadine, olanzapine, and diclofenac.
      8. The transdermal drug delivery system of any of the preceding embodiments, wherein the active pharmaceutical ingredient comprises a tertiary amine or a secondary amine.
      9. The transdermal drug delivery system of any of the preceding embodiments, wherein the adhesive is a silicone adhesive.
      10. The transdermal drug delivery system of any of the preceding embodiments, wherein the active pharmaceutical comprises a tertiary amine.
      10a. The transdermal delivery system of any of the preceding embodiments, wherein the active pharmaceutical ingredient comprises a secondary amine.
      11. The transdermal drug delivery system of any of the preceding embodiments, wherein the active layer comprises a reservoir that contains at least some of the active pharmaceutical ingredient.
      12. The transdermal drug delivery system of embodiment 11, wherein the reservoir is defined by a space between the adhesive and the backing.
      13. The transdermal drug delivery system of embodiment 11 or 12, wherein the active layer comprises a second adhesive, the second adhesive constituting a reservoir and having at least some of the active pharmaceutical ingredient dissolved or dispersed therein.
      14. The transdermal drug delivery system of any of embodiments 11-13, wherein the second adhesive has a lower affinity for the active pharmaceutical ingredient than the first adhesive.
      15. The transdermal drug delivery system of any of embodiments 11-14, wherein the active pharmaceutical ingredient diffuses through the second adhesive more slowly than it diffuses through the first adhesive.
      16. The transdermal drug delivery system of any of embodiments 11-15, wherein the transdermal drug delivery system provides controlled release of the active pharmaceutical ingredient.
      17. The transdermal drug delivery system of any of embodiments 1-10, wherein the active pharmaceutical ingredient is disposed within a matrix.
      18. The transdermal drug delivery system of any of embodiments 1-17, wherein the matrix is an adhesive.
      19. The transdermal drug delivery system of any of the preceding embodiments, wherein the pharmaceutically active ingredient is present as solid particles.
      20. The transdermal drug delivery system of any of the preceding embodiments, wherein a force needed to remove the release liner from the transdermal delivery system is 5 to 50 g/25 mm when tested in a 180° peel test at 30.5 cm/min with a 5K load.
      20a. The transdermal drug delivery system of any of the preceding embodiments, wherein a force needed to remove the release liner from the transdermal delivery system is 10 to 40 g/25 mm when tested in a 180° peel test at 30.5 cm/min with a 5K load.
      20b. The transdermal drug delivery system of any of the preceding embodiments, wherein a force needed to remove the release liner from the transdermal delivery system after storage for 4 weeks at 25° C. and 60% relative humidity is 5 to 50 g/25 mm when tested in a 180° peel test at 30.5 cm/min with a 5K load.
      20c. The transdermal drug delivery system of any of the preceding embodiments, wherein a force needed to remove the release liner from the transdermal delivery system after storage for 4 weeks at 25° C. and 60% relative humidity is 10 to 40 g/25 mm when tested in a 180° peel test at 30.5 cm/min with a 5K load.
      20d. The transdermal drug delivery system of any of the preceding embodiments, wherein a force needed to remove the release liner from the transdermal delivery system after storage for 8 weeks at 25° C. and 60% relative humidity is 5 to 50 g/25 mm when tested in a 180° peel test at 30.5 cm/min with a 5K load.
      20e. The transdermal drug delivery system of any of the preceding embodiments, wherein a force needed to remove the release liner from the transdermal delivery system after storage for 8 weeks at 25° C. and 60% relative humidity is 10 to 40 g/25 mm when tested in a 180° peel test at 30.5 cm/min with a 5K load.

20f. The transdermal drug delivery system of any of the preceding embodiments, wherein a force needed to remove the release liner from the transdermal delivery system after storage for 12 weeks at 25° C. and 60% relative humidity is 5 to 50 g/25 mm when tested in a 180° peel test at 30.5 cm/min with a 5K load.

20g. The transdermal drug delivery system of any of the preceding embodiments, wherein a force needed to remove the release liner from the transdermal delivery system after storage for 12 weeks at 25° C. and 60% relative humidity is 10 to 40 g/25 mm when tested in a 180° peel test at 30.5 cm/min with a 5K load.
20h. The transdermal drug delivery system of any of the preceding embodiments, wherein a force needed to remove the release liner from the transdermal delivery system after storage for 4 weeks at 40° C. and 75% relative humidity is 5 to 50 g/25 mm when tested in a 180° peel test at 30.5 cm/min with a 5K load.
20i. The transdermal drug delivery system of any of the preceding embodiments, wherein a force needed to remove the release liner from the transdermal delivery system after storage for 4 weeks at 40° C. and 75% relative humidity is 10 to 40 g/25 mm when tested in a 180° peel test at 30.5 cm/min with a 5K load.
20j. The transdermal drug delivery system of any of the preceding embodiments, wherein a force needed to remove the release liner from the transdermal delivery system after storage for 8 weeks at 40° C. and 75% relative humidity is 5 to 50 g/25 mm when tested in a 180° peel test at 30.5 cm/min with a 5K load.
20k. The transdermal drug delivery system of any of the preceding embodiments, wherein a force needed to remove the release liner from the transdermal delivery system after storage for 8 weeks at 40° C. and 75% relative humidity is 10 to 40 g/25 mm when tested in a 180° peel test at 30.5 cm/min with a 5K load.
20i. The transdermal drug delivery system of any of the preceding embodiments, wherein a force needed to remove the release liner from the transdermal delivery system after storage for 12 weeks at 40° C. and 75% relative humidity is 10 to 40 g/25 mm when tested in a 180° peel test at 30.5 cm/min with a 5K load
21. The transdermal drug delivery system of any of the preceding embodiments, wherein the release layer comprises at least one par by weight of the first fluorosilicone polymer per 100 parts by weight of the combined weight of second fluorinated silicone polymer and the non-fluorinated silicone polymer combined.
22. The transdermal drug delivery system of any of the preceding embodiments, wherein the release layer comprises at least 1 part by weight and no greater than 30 parts by weight of the first fluorosilicone polymer per 100 parts by weight of the combined weight of second fluorinated silicone polymer and the non-fluorinated silicone polymer combined.
23. The transdermal drug delivery system of any of the preceding embodiments, wherein the non-fluorinated silicone polymer has a vinyl equivalent weight of 1,500 to 10,000 grams per equivalent.
24. The transdermal drug delivery system of any of the preceding embodiments, wherein the release layer comprises a blend of the first fluorosilicone polymer, the second fluorosilicone polymer, and the non-fluorinated silicone polymer.
25. The transdermal drug delivery system of any of the preceding embodiments, wherein the release layer comprises a blend of:
a first fluorosilicone polymer that has neither ethylenically unsaturated groups, nor Si—H groups, nor functional groups that are chemically reactive with ethylenically unsaturated groups or Si—H groups,
   and the at least partially cured reaction product of:
a second fluorosilicone polymer that has at least two cross-linkable functional groups per polymer chain,
a non-fluorinated silicone polymer that comprises at least two cross-linkable functional groups per polymer chain and does not contain any fluorine atoms, and
a cross-linking agent.
   26 A method of delivering a drug, comprising
removing the release liner from the transdermal drug delivery system of any of the preceding embodiments; and
contacting a subject with the active layer.
   27. A method of making the transdermal drug delivery system of any of embodiments 1-25, the method comprising:

applying the active layer to on a release liner; and
laminating the backing layer on the active layer.

### EXAMPLES

The first fluorosilicone polymer used in Example lwas prepared by adding heptane (100 mL) and pentadimethyldisiloxane (13.1 g, Gelest Inc., Morrisville, Pa., USA) to 110.1 g of SYL-OFF Q2-7785 (a vinyl-functional fluorosilicone polymer formulated with a platinum hydrosilation catalyst, 88 weight percent solids in heptane, 96.9 g of polymer, approximately 32.3 mmol of vinyl functionality, Dow Corning Corp.) and heating the mixture at 60° C. overnight. The solvent and excess pentamethyldisiloxane were separated under reduced pressure to yield 100.4 g of a viscous, light amber fluid. Heptane (25.1 g) was added to the fluid to provide an 80 weight percent solids solution of the first fluorosilicone polymer. The ¹H and ²⁹Si NMR spectra indicated complete consumption of starting vinyl group functionality.

The non-fluorinated silicone polymer used in Example 1 was prepared by mixing SILMER VIN 70 (Siltech Corporation, Ontario, Canada) with 120 ppm of platinum catalyst (platinum-divinyltetramethyldisiloxane complex in xylene, Gelest Inc.) and 0.2 weight percent of the inhibitor diallyl maleate (Momentive Performance Materials Inc., Waterford, N.Y., USA).

### Example 1

The release layer for the release liner of the transdermal drug delivery system was prepared by combining a 90:10 mixture of the non-fluorinated silicone polymer and the second fluorosilicone polymer SYL-OFF 7786 (Dow Corning Corp.) with 12 pph of the first fluorosilicone polymer, crosslinker SYL-OFF 7488 (1.28:1 hydride to vinyl group, Dow Corning Corp.), and crosslinker SYL-OFF Q2-7560 (2:1 hydride to vinyl group, Dow Corning Corp.) in a solvent system of heptane:ethyl acetate (20:80). The mixture in solvent was blended to provide a coating solution with 14 weight percent solids.

The coating solution was then gravure coated onto a 2 mil Hostaphan® 3SAC primed PET film (Mitsubishi Polyester Film Inc., Greer, S.C., USA) and cured at about 116° C. to provide a coat weight of about 1.3 g/m². The resulting release liner was aged for a minimum of one week at 23° C. and 50% relative humidity.

### Example 2

Silicone adhesive BIO PSA 7-4560 (Dow Corning Corp.) was solvated in heptane at a concentration of 50 weight percent and lidocaine was blended into the solvated adhesive at a concentration of 5 weight percent. The resulting formulation was then knife coated onto the coated surface of the release liner of Example 1 using a gap of 6 mil and then dried at 82° C. for about ten minutes to provide a coat weight of about 49 gsm. The dried film was then laminated (using a soft rubber roller and light pressure) to a 1.97 mil PET film which served as the backing. The laminated product was stored at 40° C. and 75% relative humidity in a controlled environment chamber.

### Comparative Example

Silicone adhesive BIO PSA 7-4560 (Dow Corning Corp.) was solvated in heptane at a concentration of 50 weight percent and lidocaine was blended into the solvated adhesive at a concentration of 5 weight percent. The resulting formulation was then knife coated onto SCOTHPAK 9744 release liner (a perfluorinated polymer coated polyester substrate film commercially available from 3M Company) using a gap of 6 mil and then dried at 82° C. for about ten minutes to provide a coat weight of about 49 gsm. The dried film was then laminated (using a soft rubber roller and light pressure) to a 1.97 mil PET film which served as the backing. The laminated product was stored at 40° C. and 75% relative humidity in a controlled environment chamber.

### Example 3

The laminated products prepared in Example 2 and the Comparative Example were cut into 25 mm wide by 200-250 mm long samples and tested for peel force using an IMASS SP-2100 instrument (IMASS Inc., Accord, Mass., USA) with a 5 Kg load cell. Each sample was individually attached to the platen of the instrument by adhering the uncoated side of the release liner to the platen with double-sided tape. At one end of the sample, the coated backing was manually separated from the release liner, peeled back about 25 mm, and then folded onto itself The instrument clamp was then attached at one end to the folded film section and at the other end to the load cell. The 180° peel test was conducted at 30.5 cm/minute.

Samples were prepared and tested at three time points (after 4 weeks, 8 weeks, and 12 weeks of storage in the controlled environment chamber at 40° C. and 75% relative humidity). Prior to testing, all of the samples were equilibrated at 21° C. and 50% relative humidity. At each time point six samples were evaluated. The mean peel force (g/25 mm) results with standard deviation for each time point are reported in Table 1.

TABLE 1

| | Mean Peel Force in g/25 mm | | |
|---|---|---|---|
| | (standard deviation) | | |
| | 4 Weeks | 8 Weeks | 12 Weeks |
| Example 2 | 9.6 (± 0.9) | 11.2 (± 0.3) | 16.6 (± 0.6) |
| Comparative | 50.6 (± 11.1) | 68.2 (± 11.9) | 77.3 (± 6.4) |
| Example 1 | | | |

1. A transdermal drug delivery system comprising
a backing;
an active layer disposed on the backing, the active layer comprising:
an active pharmaceutical ingredient, and
an adhesive;
a release liner disposed over the active layer, the release liner comprising:
a substrate, and
a release layer disposed on the substrate; wherein
at least a portion of the release layer is in contact with the active layer;
the release layer comprising a blend of:
a first fluorosilicone polymer that has neither ethylenically unsaturated groups, nor Si—H groups, nor functional groups that are chemically reactive with ethylenically unsaturated groups or Si—H groups,
and the at least partially cured reaction product of:
a second fluorosilicone polymer that has at least two cross-linkable functional groups per polymer chain, and
a non-fluorinated silicone polymer that comprises at least two cross-linkable functional groups per polymer chain and does not contain any fluorine atoms; and
wherein the active pharmaceutical ingredient comprises at least one amine.

2. The transdermal drug delivery system of claim 1, wherein the cross-linkable functional groups of the second fluorosilicone polymer comprise ethylenically unsaturated groups.

3. The transdermal drug delivery system of claim 1, wherein the cross-linkable functional groups of the non-fluorinated silicone polymer comprise ethylenically unsaturated groups.

4. The transdermal drug delivery system of claim 1, wherein the active pharmaceutical ingredient comprises at least one of buprenorphine, clonidine, fentanyl, granisetron, methyl phenidate, oxybutynin, rivastigmine, rotigotine, scopolamine, selegiline, nicotine, sumatriptan, capsaicin, diclofenac epolamine, lidocaine, etidocaine, ropivacaine, indapamide, apomorphine, propylnorapromorphine, salbuterol, lisuride, dihydroergotamine, pergolide, terguride, proterguride, propranolol, imipramine, guanethidine, cyproheptadine, olanzapine, and diclofenac.

5. The transdermal drug delivery system of claim 1, wherein the amine is a secondary amine or a tertiary amine.

6. The transdermal drug delivery system of claim 1, wherein the adhesive is a silicone adhesive.

7. The transdermal drug delivery system of claim 1, wherein the active layer comprises a reservoir that contains at least some of the active pharmaceutical ingredient.

8. The transdermal drug delivery system of claim 1, wherein the active pharmaceutical ingredient is disposed within a matrix.

9. The transdermal drug delivery system of claim 1, wherein the release layer comprises 65 to 97 weight percent of the non-fluorinated silicone polymer based on the combined weight of the non-fluorinated silicone polymer and the second fluorinated silicone polymer.

10. The transdermal drug delivery system of claim 1, wherein the release layer comprises at least 1 part by weight and no greater than 30 parts by weight of the first fluorosilicone polymer per 100 parts by weight of the second fluorinated silicone polymer and the non-fluorinated silicone polymer combined.

11. The transdermal drug delivery system of claim 3, wherein the non-fluorinated silicone polymer has a vinyl equivalent weight of 1,500 to 10,000 grams per equivalent.

12. The transdermal drug delivery system of claim 1, wherein a force needed to remove the release liner from the transdermal delivery system is 5 to 50 g/25 mm when tested in a 180° peel test at 30.5 cm/min with a 5Kg load.

13. The transdermal drug delivery system of claim 1, wherein the release layer comprises a blend of:
a first fluorosilicone polymer that has neither ethylenically unsaturated groups, nor Si—H groups, nor functional groups that are chemically reactive with ethylenically unsaturated groups or Si—H groups,
and the at least partially cured reaction product of:
a second fluorosilicone polymer that has at least two cross-linkable functional groups per polymer chain,
a non-fluorinated silicone polymer that comprises at least two cross-linkable functional groups per polymer chain and does not contain any fluorine atoms, and
a cross-linking agent.

14. A method of delivering a drug, comprising
removing the release liner from the transdermal drug delivery system of claim 1; and
contacting a subject with the active layer of the transdermal drug delivery system.

15. A method of making the transdermal drug delivery system of claim 1, the method comprising:
applying the active layer to on a release liner; and
laminating the backing layer on the active layer.
